(19) United States
(12) Patent Application Publication
(10) Pub. No.: US 2013/0018140 A1
(43) Pub. Date: Jan. 17, 2013

(54) N-BRIDGED POLYNITRONS AND THE USE THEREOF AS CROSS-LINKING AGENTS, IN PARTICULAR IN COATING MATERIALS AND ADHESIVES

(71) Applicants: Marcus Dickmeis, Krefeld (DE); Hakan Cinar, Neuss (DE); Helmut Ritter, Wuppertal (DE)

(72) Inventors: Marcus Dickmeis, Krefeld (DE); Hakan Cinar, Neuss (DE); Helmut Ritter, Wuppertal (DE)

(21) Appl. No.: 13/520,070

(22) PCT Filed: Dec. 21, 2010
(86) PCT No.: PCT/EP10/07848

§ 371 Date: Oct. 5, 2012

(30) Foreign Application Priority Data
Dec. 24, 2009 (DE) 10 2009 060 968.7

(51) Int. Cl. C07C 249/04; C07D 401/12; C09D 131/00; C09D 11/10; C08G 73/00; C08L 31/00

(52) U.S. Cl. 524/512; 528/245; 525/154; 564/265; 546/264

(57) ABSTRACT

The invention relates to N-bridged polynitrons and their use for cross-linking of unsaturated polymers, as well as a curable composition comprising (a) a N-bridged polynitron, (b) an unsaturated polymer or a mixture of polymers, wherein at least one unsaturated polymer contains unsaturated functions or functional groups which can react with the polynitron, (c) optionally fillers, (d) optionally pigments, (e) optionally additives such as plasticizers, stabilizers, photoinitiators, and (f) optionally, other cross-linking agents such as polyisocyanates, bisdienes, polyoxaziridines, and their use as adhesives, rubber, filling, sprayable thick-layer filler, ink, paint, powder paint, waterborne paint or solvent based paint. Further, the invention relates to cross-linking products obtainable by curing the curable composition according to the present invention.

The invention relates to the area of cross-linking agents for polymers. These are molecules which contain more than two functional groups, with the help of which linear or branched macromolecules can be connected with each other to three-dimensional networks. For this purpose, multifunctional N-bridged nitrons are proposed.

Cross-linking agents have many technical applications, since, for instance, the mechanical properties of polymers strongly depend on the degree of cross-linking. Of particular importance are cross-linking agents in the production of solid coatings from paint formulations. In the paint technology, the cross-linking agent is generally regarded as component, which has the lower molecular weight and is present in a smaller amount. Cross-linking agents have a strong influence on the production, storage, application and processing properties of the coatings such as extrusion temperature, storage stability, curing time, curing temperature, flow properties, impact strength, hardness, weather resistance and chemical resistance. Examples of cross-linked polymers are paints.

Coatings can be classified according to the solvent used in solvent based paints, waterborne paints and powder paints. Modern paints are increasingly solvent reduced or even solvent-free and thus reduce the amount of volatile organic solvents (VOCs), which are emitted into the environment. This helps to reduce the amount of waste and prevent people being exposed to critical air mixtures. For these reasons, solvent based systems are increasingly substituted by environmentally friendly paints such as powder paints.

There is a substantial limitation in the application of conventional powder paints because they need high temperatures of 160 to 230° C. for the curing. These high firing temperatures lead to the fact that only substrates with high heat resistance can be used. The coating of wood and plastics, but also of metal alloys with special properties is therefore only possible to a limited extent with thermally curable powder paints. Therefore, the demand for low-temperature powder paints that cure at low temperature is very high.

The curing of powder paints mostly occurs by reaction of an epoxide with an acid, an acid anhydride, an amine, a phenol or a Lewis base or by addition of an isocyanate to an alcohol. Most commercially available cross-linking agents therefore contain identical reactive ends, such as epoxides, amines, phenols or isocyanates. In addition, reference is made to Powder Coatings Chemistry and Technology by Pieter Gillis de Lange, Vincentz Network 2003.

Paints, particularly powder paints often contain unsaturated polymers as binders. For example, unsaturated polyesters or acrylates are used. Cross-linking agents that induce cross-linking via unsaturated functions are not well known. Curing by radical polymerization (e.g. via styrene) requires initiators and mostly harmful heavy metal catalysts. It also has only practical significance for radiation curable paints. The generation of radicals is effected by an initiator which decomposes into free radicals. Radiation curable paints have a number of unsolved problems that often result from the limited thickness of layers. This limitation is caused in that the UV radiation has to completely penetrate the substrate. For example, many techniques thermally curable paints are structured and modified with, for example, with pigments, fillers or solid additives can only be used to a limited extent. In addition, the uniform curing of three-dimensional edges is a problem.

In addition to the cross-linking of unsaturated polymers by radical polymerization, a cross-linking via cycloaddition is known, which requires no initiators and harmful heavy metal catalysts.

WO 2009/074310 A1 describes polynitrons, and in particular C-bridged polynitrons as evidenced by the general formula stated therein and their use as a networking and matting agent, preferably for the production of stable moldings, filling compounds, their use in paints, varnishes and adhesives. This cross-linking reaction takes place at relatively low firing temperature by cycloaddition between polynitron and unsaturated polymer. The polynitrons according to WO 2009/074310 A1 are synthesized by reacting a dialdehyde with N-alkyl- or N-arylhydroxylamine. The variation of the structure of polynitrons takes place mostly on a change in the structure of the dialdehydes. These dialdehydes must generally first be synthesized in several steps.

Furthermore, due to the limited accessibility of hydroxylamines the synthesis is limited (H. Mitsui, S.-I. Zenki, T. Shiota, S. Murahashi, Journal of the Chemical Society, Chemical Communications 1984, 874-875). Only a few hydroxylamines such as N-methylhydroxylamine hydrochloride and benzyl hydroxylamine hydrochloride are commercially available, which limits the variation of the polynitron structure. Thus, the properties of polynitrons such as melting point and the ones of the cross-linking products such as glass temperature can be varied only to a limited extent. For example, polynitrons which reduce the glass temperature of the unsaturated polymer by cross-linking, are hardly produced. In addition, the high costs of hydroxylamines complicate an economic large-scale application.

In the area of expertise the cycloaddition of monofunctional nitrons to unsaturated monofunctional low molecular weight compounds such as alkynes, alkenes and heterocumulenes is known. The cycloaddition of N-linked polynitrons to unsaturated polymers is unknown in the area of expertise.

The object of the present invention was to find new cross-linking agents that exhibit the positive properties of the C-bridged polynitrons without exhibiting their disadvantages discussed above.

Therefore, one embodiment of the invention is the use of N-linked polynitrons for the cross-linking of unsaturated polymers.

A further embodiment of the invention are curable compositions, comprising

(a) a N-bridged polynitron,

(b) an unsaturated polymer or a mixture of polymers, wherein at least one unsaturated polymer contains functions or functional groups which can react with the N-bridged polynitron,

(c) optionally fillers and

(d) optionally pigments

(e) optionally additives such as plasticizers, stabilizers or photoinitiators

(f) optionally further cross-linking agents such as polyisocyanates, bisdienes, polyoxaziridines.

Nitrons are usually compounds with the structural element

C(R1)(R2)═NO(R3)

wherein the double bond is between C and N and the radical R3 is bound to the nitrogen, and wherein the radicals R1, R2 and R3 are hydrogen or any alkyl or aryl radicals which may also be substituted, under the proviso that at least one radical is not hydrogen.

According to the invention N-bridged polynitrons are used. According to the invention these compounds are understood to be compounds whose molecules contain more than one, preferably 2 to 20 nitron groups, especially 3 to 6 nitron groups, which are bound to each other via the N-side, i.e. via the radical R3.

The production of mono-functional nitrons is known in the art and was transferred within the framework of this invention to N-bridged polynitrons. Starting compounds are polyimines that are converted under conditions that are familiar to the organic chemist, to N-bridged polynitrons. Polyimines in turn can be obtained by reaction of polyfunctional aldehydes or ketones with monofunctional primary amines, or of polyfunctional primary amines with monofunctional aldehydes or ketones. For the purposes of the invention N-bonded polynitrons from polyfunctional amines are strictly preferred. These compounds are defined as N-linked polynitrons due to their structure.

The present invention thus relates to a process for preparing polynitrons and preferably N-bridged polynitrons from polyimines of a functionality of 2 and higher, preferably from 2 to 20 and particularly preferably from 3 to 6.

It is advantageous that primary amines and carbonyl compounds are commercially available in countless variations which results in almost unlimited variation possibilities of the N-bridged polynitron structures.

N-bridged polynitrons according to the present invention may contain aromatic radicals, including any, and also substituted radicals having at least one aromatic group. Suitable aromatic radicals may include heteroatoms such as nitrogen, sulfur, selenium, silicon and oxygen or may be exclusively composed of carbon and hydrogen. The term “aromatic radical” (or “aromatic residue”), as used herein includes, without limitation, phenyl, pyridyl, furanyl, thienyl, naphthyl, phenylene, and biphenyl radicals. As indicated, the aromatic radical contains at least one aromatic group. The aromatic group invariably is a cyclic structure having 4n+2 “delocalized” electrons where “n” is an integer equal to 1 or more, as illustrated by phenyl groups (n=1), thienyl (n=1), furanyl groups (n=1), naphthyl groups (n=2), azulenyl groups (n=2), anthracenyl groups (n=3) and the like. The aromatic radical may also include non-aromatic components. Thus, for example, a benzyl group is an aromatic radical comprising a phenyl ring (the aromatic group) and a methylene group (the non-aromatic component). Similarly, a tetrahydronaphthyl radical is an aromatic radical, comprising an aromatic group (C₆H₃), fused to a non-aromatic component —(CH₂)₄—.

For the sake of simplicity, the term “aromatic radical” is defined herein as comprising a wide range of functional groups, such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (e.g. carboxylic acid derivatives such as esters and amides), amine groups, nitro groups, sulfonyl groups, sulfamyl, phosphinoyl and the like. Thus, for example, the 4-methylphenyl radical is an aromatic C₇-radical comprising a methyl group, the methyl group is a functional group which is an alkyl group. Similarly, the 2-nitrophenyl group is an aromatic C₆-radical, comprising a nitro group, the nitro group is a functional group. Aromatic radicals include halogenated aromatic radicals such as 4-trifluoromethylphenyl, hexafluoroisopropylidenbis(4-phen-1-yloxy) (i.e., —OPhC (CF₃)₂PhO—), 4-chloromethylphen-1-yl, 3-trifluorovinyl-2-thienyl, 3-trichloromethylphen-1-yl (i.e., 3-CCl₃Ph-), 4-(3-bromoprop-1-yl)phen-1-yl (i.e., 4-BrCH₂CH₂CH₂Ph-) and the like. Further examples of aromatic radicals include 4-allyloxyphen-1-oxy, 4-aminophen-1-yl (i.e., 4-H₂NPh-), 3-aminocarbonyl-phen-1-yl (i.e., NH₂COPh-), dicyanomethylidenbis (4-phen-1-yloxy) (CN)₂PhO—), 4-benzoylphen-1-yl, 3-methylphen-1-yl, methylenebis(4-phen-1-yloxy) (i.e., —OPhCH₂PhO), 2-ethylphen-1-yl, phenylethenyl, fluorenyl, 3-formyl-2-thienyl, 2-hexyl-5-furanyl, hexamethylene-1,6-bis(4-phen-1-yloxy) (i.e., —OPh(CH₂)₆PhO—), benzenesulfonyl (i.e., PhSO₂—), 4-hydroxymethylphen-1-yl (i.e., 4-HOCH₂Ph-), 4-mercaptomethylphen-1-yl (i.e., 4-HSCH₂Ph-), 4-methylthiophene-1-yl (i.e., 4-CH₃SPh-), 3-methoxyphen-1-yl, 2-methoxycarbonylphen-1-yloxy (e.g. methylsalicyl), 2-nitromethylphen-1-yl (i.e., 2-NO₂CH₂Ph-), 3-trimethylsilylphen-1-yl, 4-t-butyldimethylsilylphenyl-1-yl, 4-vinylphen-1-yl, vinylidenebis(phenyl) and the like. The term “an aromatic C₃-C₁₀-radical” includes aromatic radicals which comprise at least three but no more than 10 carbon atoms. The aromatic radical 1-imidazolyl (C₃H₂N₂—) represents an aromatic C₃-radical. The benzyl radical (C₇H₇—) represents an aromatic C₇ radical.

The N-linked polynitrons according to the present invention may also contain cycloaliphatic radicals.

The term “cycloaliphatic radical” (or “cycloaliphatic residue”) as used herein, refers to a radical having a valence of at least 1 and comprises a number of atoms which are cyclic but not aromatic. As defined herein, a “cycloaliphatic radical” contains no aromatic group. A “cycloaliphatic radical” may comprise one or more non-cyclic components. For example, a cyclohexylmethyl group (C₆H₁₁CH₂—) is a cycloaliphatic radical, which comprises a cyclohexyl ring (the number of atoms, which are cyclic but not aromatic) and a methylene group (the non-cyclic component). The cycloaliphatic radical may include heteroatoms such as nitrogen, sulfur, selenium, silicon and oxygen, or it may be composed exclusively of carbon and hydrogen. For the sake of simplicity, the term “cycloaliphatic radical” is defined herein as comprising a wide range of functional groups, such as alkyl groups, alkenyl groups, alkynyl groups, haloalkyl groups, conjugated dienyl groups, alcohol groups, ether groups, aldehyde groups, ketone groups, carboxylic acid groups, acyl groups (e.g., carboxylic acid derivatives, such as esters and amides), amine groups, nitro groups, sulfonyl groups, sulfamyl, phosphinoyl and the like. Thus, for example, the 4-methylcyclopent-1-yl radical is a cycloaliphatic C₆-radical comprising a methyl group, wherein the methyl group is a functional group which is an alkyl group. Similarly, the 2-nitrocyclobut-1-yl radical is a cycloaliphatic C₄-radical, comprising a nitro group, the nitro group is a functional group. A cycloaliphatic radical may comprise one or more halogen atoms, which may be identical or different. Halogen atoms include, for example, fluorine, chlorine, bromine and iodine. Cycloaliphatic radicals having one or more halogen atoms include 2-trifluoromethylcyclohex-1-yl, 4-bromodifluoromethylcyclooct-1-yl, 2-chlorodifluoromethylcyclohex-1-yl, hexafluoroisopropylidene-2,2-bis(cyclohex-4-yl) (i.e., —C₆H₁₀C(CF₃)₂C₆H₁₀—), 2-chloromethylcyclohex-1-yl, 3-difluoromethylene-cyclohex-1-yl, 4-trichloromethylcyclo-hex-1-yloxy, 4-bromodichloromethylcyclohex-1-ylthio, 2-bromoethylcyclopent-1-yl, 2-bromopropylcyclohex-1-yloxy (e.g. CH₃CHBrCH₂C₆H₁₀O—) and the like. Further examples of cycloaliphatic radicals include 4-allyloxycyclohex-1-yl, 4-aminocyclohex-1-yl (i.e., H₂C₆H₁₀—), 4-aminocarbonylcyclopent-1-yl (i.e., NH₂COC₅H₈—), 4-acetyloxycyclohex-1-yl, 2,2-dicyanisopropylidenbis(cyclohex-4-yloxy) (i.e., —OC₆H₁₀C(CN)₂C₆H₁₀O—), 3-methylcyclohex-1-yl, methylenebis(cyclohex-4-yloxy) (i.e., —OC₆H₁₀CH₂C₆H₁₀O—), 1-ethylcyclobut-1-yl, 3-formyl-2-tetrahydrofuranyl, 2-hexyl-5-tetrahydrofuranyl, cyclopropylethenyl, hexamethylene-1,6-bis(cyclohex-4-yloxy) (i.e., —OC₆H₁₀(CH₂)₆C₆H₁₀O—), 4-hydroxymethylcyclohex-1-yl (i.e., 4-HOCH₂C₆H₁₀—), 4-mercaptomethylcyclohex-1-yl (i.e., 4-HSCH₂C₆H₁₀—), 4-methylthiocyclohex-1-yl (i.e., 4-CH₃SC₆H₁₀—), 4-methoxycyclohex-1-yl, 2-methoxycarbonylcyclohex-1-yloxy(2-CH₃OCOC₆H₁₀—), 4-nitromethylcyclohex-1-yl (i.e., NO₂CH₂C₆H₁₀—), 3-trimethylsilylcyclohex-1-yl, 2-tert-butyldimethylsilylcyclopent-1-yl, 4-trimethoxysilylethylcyclohex-1-yl (e.g. (CH₃O)₃SiCH₂CH₂C₆H₁₀—), 4-vinyl cyclohexene-1-yl, vinylidenebis(cyclohexyl), and the like. The term “a cycloaliphatic C₃-C₁₀-radical” includes cycloaliphatic radicals which contain at least three, but no more than 10 carbon atoms. The cycloaliphatic radical 2-tetrahydrofuranyl (C₄H₇O—) represents a cycloaliphatic C₄-radical. The cyclohexylmethyl radical (C₆H₁₁CH₂) represents a cycloaliphatic C₇ radical.

The N-linked polynitrons according to the present invention may also contain aliphatic radicals. The term “aliphatic radical” (or “aliphatic residue”) as used herein, refers to an organic radical having a valence of at least 1, consisting of a linear or branched array of atoms which is not cyclic. Aliphatic radicals are defined as comprising at least one carbon atom. The aliphatic radical comprising array of atoms can include heteroatoms such as nitrogen, sulfur, silicon, selenium, and oxygen, or may exclusively be composed of carbon and hydrogen. For the sake of convenience, the term “aliphatic radical” is defined herein as comprising as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (e.g., carboxylic acid derivatives such as esters and amides), amine groups, nitro groups, sulfonyl groups, sulfamyl, phosphinoyl group and the like. For example, the 4-methylpent-1-yl radical is an aliphatic C₆-radical comprising a methyl group, the methyl group is a functional group which is an alkyl group. Similarly, the 4-nitrobut-1-yl group is an aliphatic C₄-radical comprising a nitro group, the nitro group is a functional group. An aliphatic radical can be a haloalkyl group, comprising one or more halogen atoms which may be the same or different. Halogen atoms comprise, e.g., fluorine, chlorine, bromine, and iodine. Aliphatic radicals comprising one or more halogen atoms comprise the alkyl halides trifluoromethyl, bromodifluoromethyl, chlorodifluoromethyl, hexafluoroisopropylidene, chloromethyl, difluorovinyliden, trichloromethyl, bromodichloromethyl, bromoethyl, 2-bromotrimethylen (e.g. —CH₂CHBrCH₂—) and the like. Further examples of aliphatic radicals include allyl, aminocarbonyl (i.e., —CONH₂), carbonyl, 2,2-dicyanisopropyliden (i.e., —CH₂C(CN)₂CH₂—), methyl (i.e., —CH₃), methylene (i.e., —CH₂—), ethyl, ethylene, formyl (i.e., —CHO), hexyl, hexamethylene, hydroxymethyl (i.e., —CH₂OH), mercaptomethyl (i.e., —CH₂SH), methylthio (i.e., —SCH₃), methylthiomethyl (i.e., —CH₂SCH₃), methoxy, methoxycarbonyl (i.e., CH₃OCO—), nitromethyl (i.e., —CH₂NO₂), thiocarbonyl, trimethylsilyl (i.e., (CH₃)₃Si—), tert-butyldimethylsilyl, 3-trimethyloxysilylpropyl (i.e., (CH₃O)₃SiCH₂CH₂CH₂—), vinyl, vinylidene and the like. As another example, an aliphatic C₁-C₁₀ radical contains at least one but not more than 10 carbon atoms. A methyl group (i.e., CH₃) is an example of an aliphatic C₁-radical. A decyl group (i.e., CH₃(CH₂)₉—) is an example of an aliphatic C₁₀ radical.

Preferred N-bridged polynitrons correspond to a general formula

[X—Z(—X)n]m

in which X is an optionally substituted via the nitrogen atom bonded nitro group, Z is an intermediate group with n−1 binding equivalences and n and m is an integer >0.

Preferably Z is a polyfunctional aliphatic, cycloaliphatic, aromatic, substituted or unsubstituted intermediate group which may also contain hetero atoms and/or may be a polymer, and n and m is an integer greater than 1.

Preferred intermediate groups Z are derived from the following structures:

Mono- or polynuclear aromatic, cycloaliphatic groups, or alkyl chains. The groups can be substituted as desired. Particularly suitable are the following compounds or combinations of the following compounds, wherein these can be linked with each other arbitrarily, in particular via an ester, amide, ether, urea or urethane function:

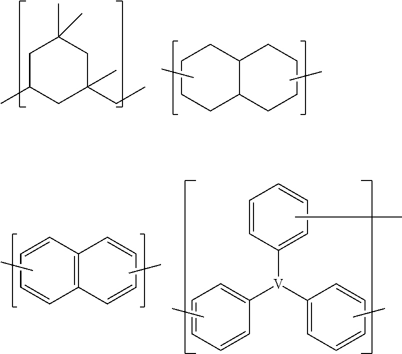

Therein, V is preferably a group selected from methine, nitrogen, phosphorous, phosphine oxide.

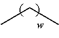

W is a natural number greater than zero, preferably six.

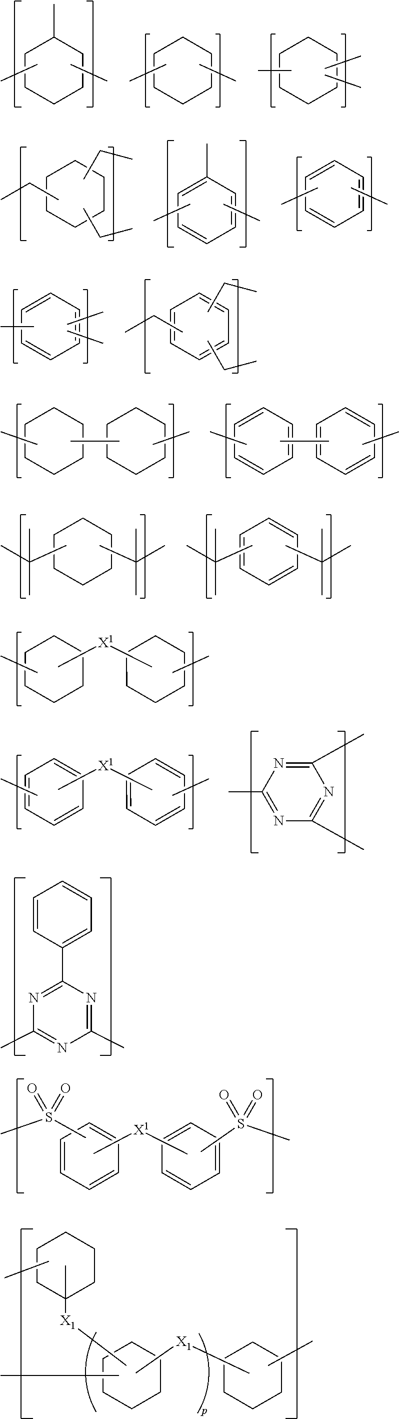

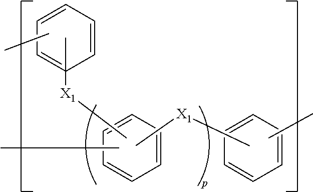

Therein, p is a natural number greater than or equal to 1.

X¹ is preferably an aliphatic, cycloaliphatic, aromatic, substituted or unsubstituted group which may also contain hetero atoms and/or may be a polymer. More preferably X¹ is a group selected from methylene, oxygen, sulfur, carbonyl, sulfone, tertiary amine, alkylene, arylene, and the following groups. Y is a natural number greater than one, preferably six or four.

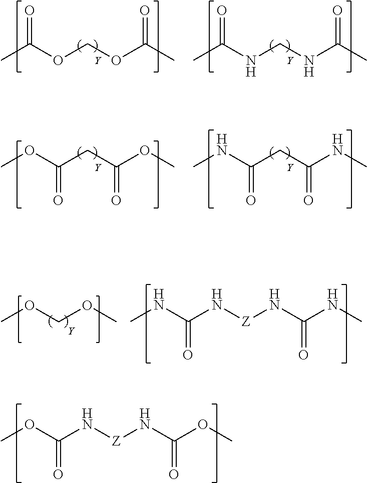

Particularly preferred intermediate groups Z have the following structure:

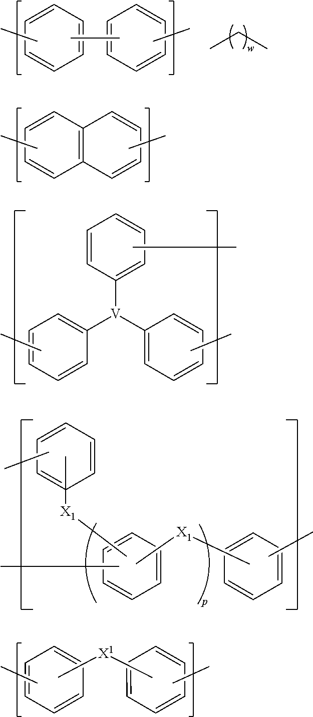

According to a particularly preferred embodiment of the invention, the substituents at the C atom of the nitro group form a ring (particularly a cyclohexane, cyclopentane-fluoren-, cyclohexa-2,4-dienone, cyclohexa-2,5-dienone, anthracene-10-one-, phenanthrene-9-one-, naphthalene-1-one-, naphthalene-2-one ring). According to a further embodiment, the substituents at the C atom of the nitron independently from each other are hydrogen, an aryl group (especially phenyl, naphthyl), a heteroaryl group (especially pyridinyl, furyl, thienyl, thiazolyl or benzothiazolyl), an alkyl group, a cycloalkyl group or a combination thereof. According to a further embodiment, the substituent of the nitro group themselves form a ring.

The novel N-bridged polynitrons are preferably prepared by oxidation of a polyimine. Optionally, the polyimine is previously hydrogenated to the polyamine (reductive amination) and only subsequently oxidized. The hydrogenation is preferably carried out with hydrogen gas in the presence of a suitable catalyst such as palladium, platinum or nickel. Alternatively, for hydrogenation, hydrides such as sodium borohydride can be used or hydrogen donors such as ammonium formate, formic acid, cyclohexenes, cyclohexadienes or hydrazides (catalytic transfer hydrogenation).

The polyimine can be produced by reaction of a monofunctional aldehyde or a ketone with a polyfunctional amine. This can be made in situ where appropriate. In contrast to the condensation of an amine with an aldehyde, in the reaction of an amine with a ketone, a Bronsted or Lewis acid catalyst (e.g., titanium tetrachloride) is often required. Without that the process of the invention is limited thereto, the following starting materials can be used for the synthesis of the polymines:

As aldehydes: formaldehyde, vanillin, acetaldehyde, propionaldehyde, butyraldehyde, isobutyraldehyde, Oenantal, ethyl-2-hexanal, cyclohexanecarbaldehyde cyclopentanecarbaldehyde, hexahydrobenzaldehyde, benzaldehyde, o-, m-, p-anisaldehyde, salicylaldehyde, p-tolualdehyde, monochlorobenzaldehyd, o-, m-, p-nitrobenzaldehyde, o-, m-, p-amino aldehyde, o-, m-, p-dimethylaminobenzaldehyde, beta-methoxypropionaldehyde, beta-ethyloxypropionaldehyde, malondialdehyde, glyoxal, glycolaldehyde, glyceraldehyde, succinic, malonic, glutaric, adipic aldehydes, 1,10-di(4-formylphenoxy)decane, 1,4-di(4-formylphenoxy)butane, 1,6-di(4-formylphenoxy)hexane, 1,10-di(4-formylbenzoate decane), 1,4-di(4-formylbenzoate)butane, 10-di(4-formylbenzoate)hexane, bis(4-formylphenyl)succinate, bis(4-formylphenyl)glutarate, bis(4-formylphenyl)heptanedioate, bis(4-formylphenyl)adipate, bis(4-formylphenyl)nonadioate, bis(4-formylphenyl)decanedioate, citronellal, N¹,N¹⁰-bis(4-formylphenyl)decandiamid, N¹,N⁹-bis(4-formylphenyl)nonadiamide, crotonaldehyde, N¹,N⁷-bis(4-formylphenyl)heptanediamide, N¹,N⁶-bis(4-formylphenyl)adipamide, N¹,N⁴-bis(4-formylphenyl)succinamide, terephtalaldehyde, phthalaldehyde, isophthalaldehyde, acrolein, lsochrotonaldehyde, indole-3-carbaldehyde, tris(4-formylphenyl)amine, tris(4-formylphenyl)methane, 4-methoxynaphthalen-1-carbaldehyde, 4-(4-formylphenoxy)benzaldehyde, citral, 4-(4-formylphenylthio)benzaldehyde, 2,4-dimethoxybenzaldehyde, etc. . . .

As ketones: acetone, 2-butanone, 2-pentanone, 3-pentanone, methylisopropyl ketone, diisopropyl ketone, benzyl methyl ketone, ethyl methyl ketone, 3-oxohexanoic acid, methyl isobutyl ketone, methyl cyclohexyl ketone, acetophenone, benzophenone, cyclobutanone, cyclopentanone, cyclohexanone, methyl-2-cyclohexanone, methyl-3-cyclohexanone, methyl-4-cyclohexanone, dimethyl-2,4-cyclohexanone, methyl-4-cyclohexanone, dimethyl-2,4-cyclohexanone, trimethyl-3,3,5-cyclohexanone, cycloheptanone, cyclooctanone, cyclodecanone, cyclododecanone, cyclohexandiketon-1,4, isophorone, 9-fluorenone, p-benzoquinone, o-benzoquinone, 1,4-, 1,2- and 2,6-naphthoquinone, 9,10-anthraquinone, 9,10-phenanthrenequinone, toluquinone, fumigatin, phtiocol, alizarin, junglon, rhein, 2,3-butanedione, 1,2-diphenyl-1,2-ethane, 2,4-pentanedione, menthone, carvone, camphor etc. . . .

As diamines: ethylenediamine, diethylenetriamine, triethylenetetramine, diaminoalkanes, such as 1,2-propylenediamine, 1,3-propanediamine, 1,6-diaminohexane, diamino-pyridine, 1,8-diaminooctane, 1,5-diaminopentane or 1,4-diaminobutane, 1,5-diamino-2-methyl-pentane, 2,2-dimethyl-1,3-propanediamine, phenylenediamines, diaminocyclohexanes, toluene diamines, diaminodiphenylmethanes, methylene bis(cyclohexylamine), 4-aminophenyl sulfone, isophorone diamine, diamino-naphthalenes, melamine, benzoguanimine, 1,3,5-tris(aminomethyl)cyclohexane, 1,3,5-tris(aminomethyl)benzene, m- and p-tetramethylxyloldiamine, polyethyleneimine, dicyandiamide, polymeric diphenylmethane diamine, polymeric methylene-bis(cyclohexylamine), 3,3′-dimethyl-4,4′-diamino-dicyclohexyl methane, tetramethylbenzidine, o-, m- and p-diaminodiphenyl, lysine, 4-(4-aminophenoxy)benzenamine, benzidine, diphenyline, 4-(4-aminophenylthio)benzenamine, 1,5-decahydronnaphtylenediamine, 1,8-diamino-p-menthane, diamino anthraquinones, diamino benzenesulfonic, 1,5-diamino-anthraquinone, etc. . . .

According to another particularly preferred embodiment of the invention, the inventive cross-linking agents are based on polymers having primary amine groups, for example of polyvinyl amines, polyamides, polyurethanes, urea melamine resins or modified copolymers of other origin which are then reacted with aldehydes or ketones to give the N-bridged polynitrons.

The oxidizing agent is preferably selected from, for example,

(i) molecular oxygen (in combination with a suitable catalyst),

(ii) peracids, particularly preferably meta-chloroperbenzoic acid,

(iii) dimethyldioxirane, particularly preferably prepared by reaction of acetone with oxone,

(iv) potassium permanganate,

(v) hydrogen peroxide, also in form of more complex systems such as nitrile-hydrogen peroxide systems

(vi) hydrogen peroxide in the presence of a suitable catalyst, preferably selenium dioxide or sodium tungstate

(vii) alkyl hydroperoxides, such as e.g. cumene hydroperoxide or tert-butyl hydroperoxide in the presence of a suitable catalyst, more preferably titanium isopropoxide

(viii) and other suitable oxidants.

The above N-bridged polynitrons are used for cross-linking of unsaturated polymers. The term “unsaturated polymer” usually defines a polymer with one or more unsaturated carbon-carbon bonds in the polymer chain.

The degree of unsaturated carbon-carbon bonds can be determined by DIN53241 and expressed by the unit “meq/g”. Usually, the unsaturated polymers have 0.1 to 50, preferably 1 to 20 meq/g.

The unsaturated polymers are preferably selected from alkyd resin, acrylic ester-styrene-acrylonitrile copolymer, acrylonitrile-butadiene-acrylate copolymer, acrylonitrile-butadiene-styrene copolymer, polyethylene-styrene copolymer, acrylonitrile-methyl methacrylate copolymer, butadiene rubber, butyl rubber, casein plastic, artificial horn, cellulose acetate, cellulose hydrate, cellulose nitrate, chloroprene rubber, chitin, chitosan, cyclo-olefin copolymer, epoxy resin, ethylene-propylene copolymer, ethylene-propylene-diene rubber, ethylene vinyl acetate, fluorine rubber, liquid crystal polymers, urea-formaldehyde resin, isoprene rubber, lignin, melamine-formaldehyde resin, natural rubber, melamine-phenol-formaldehyde resin, methyl acrylate-butadiene-styrene copolymer, phenol-formaldehyde resin, perfluoroalkoxyalkane, polyacetal, polyacrylate, polyacrylonitrile, polyamide, polyalkylene glycol, polybenzimidazole, polybutylene succinate, polycaprolactone, polycarbonate, polychlorotrifluoroethylene, polyester, polyester amide, polyester acrylate, polyether block amide, polyether imide, polyether ketone, polyether sulfone, polyethylene, polyethylene terephthalate, polyurea, polyhydroxyalkanoate, polyhydroxybutyrate, polyimide, polyisobutylene, polyisocyanate, polyketone, polylactide, polymethacrylmethylimide, polymethylenterephtalate, polymethacrylate, polymethylpentene, polyolefin, polyoxymethylene, polysaccharide, polyphenylene ether, polyphenylene sulfide, polyphthalamide, polypropylene, polypropylene oxide, polypyrrole, polysiloxane, polystyrene, polysulfone, polytetrafluoroethylene, polyurethane, polyurethane acrylate, polyvinyl alcohol, polyvinyl acetate, polyvinyl butyral, polyvinyl chloride, polyvinyl ethers, polyvinylidene fluoride, polyvinyl pyrrolidone, silicone rubber, styrene-acrylonitrile copolymer, styrene-butadiene rubber, starch, vinyl chloride-ethylene copolymer, vinyl chloride-ethylene-methacrylate copolymer, unsaturated polyesters, polymer-bound para-toluenesulfonic acid, polymer-bound para-toluenesulfonamide, polyurethane acrylate, propargyl acrylate copolymers, cellulose, gelatin or combinations thereof, insofar as these polymers have C—C double and/or C—C triple bonds or have been modified with these.

Particularly preferably used are unsaturated polyesters, unsaturated polyester urethanes and/or polyester-urethane acrylates or polyester-urethane methacrylates, such as described for example in U.S. Pat. No. 6,284,321 B1.

Within the scope of the invention it is particularly preferred to crosslink unsaturated polyesters. Suitable unsaturated polyesters are generally considered polycondensation products of α,β-ethylenically unsaturated dicarboxylic acids such as maleic acid, fumaric acid, itaconic acid, mesaconic acid and citraconic acid, with polyalcohols such as ethylene glycol, diethylene glycol, polyethylene glycol, propane-, butane-, butene-, butyne- and hexanediols, trimethylolpropane and pentaerythritol, which may optionally contain radicals of saturated carboxylic acids, e.g. succinic acid, glutaric acid, adipic acid, phthalic acid, tetrachlorophthalic, further monofunctional alcohols such as butanol, tetrahydrofurylalcohol and ethylene glycol monobutyl ether, as well as monobasic acids such as benzoic acid, oleic acid, linseed oil fatty acid and dehydrated castor acid.

Suitable monomeric unsaturated compounds which can be copolymerized with the unsaturated polyesters are, for example, vinyl compounds such as styrene, vinyl toluene and divinyl benzene, further vinyl esters such as vinyl acetate, further unsaturated carboxylic acids and their derivatives, such as methacrylic acid, -ester and -nitrile, further allyl esters, such as allyl acetate, allyl acrylate, phthalic acid diallyl ester, triallyl phosphate and triallyl cyanurate.

In particular unsaturated polyesters are used which contain maleate and fumarate groups.

The unsaturated polymers typically have a weight average molecular weight of 200 to 500,000 g/mol, preferably from 1,000 to 200,000 g/mol, particularly from 10,000 to 100,000 g/mol.

The use of the above-described N-bridged polynitrons according to the present invention may be performed within the framework of a curable composition. This may comprise:

(a) a N-bridged polynitron,

(b) an unsaturated polymer or a mixture of polymers, wherein at least one polymer exhibits unsaturated functions or functional groups which can react with the N-bridged polynitron,

(c) optionally fillers and

(d) optionally pigments

(e) optionally additives such as plasticizers, stabilizers or photoinitiators

(f) optionally further cross-linking agents such as polyisocyanates, bisdienes, polyoxaziridines.

Basically, two preferred embodiments of the inventive curable composition are possible.

In a first embodiment, the inventive curable composition is a 2-component system. This means that the components (a) and (b) are in the form of two compounds. Thus, the components (a) and (b) are separate compounds that are not covalently linked before the onset of curing.

Basically for this first embodiment of the inventive curable composition, the explanations of the above-mentioned preferred N-bridged polynitrons are applicable. However, it is preferred that N-bridged polynitrons are used according to general formula I, wherein none of the substituents of the nitron group is bound to a polymer, in particular to an unsaturated polymer.

Also, for this first embodiment, the explanations of the above-mentioned preferred unsaturated polymers are applicable.

In this first embodiment of the inventive curable composition the N-bridged polynitron (a) is contained in an amount of 0.1 to 50 weight-%, more preferably from 1 to 20 weight-%, particularly from 5 to 15 weight-%, based on the total weight of the composition.

In a second embodiment of the inventive curable composition the curable composition is a 1-component system. This means that the components (a) and (b) are present in the form of an unsaturated polymer having more than one terminated nitron group. Thus, the components (a) and (b) are combined within a compound.

Basically, for this second embodiment of the inventive curable composition, the explanations of the above-mentioned preferred N-bridged polynitrons are applicable. However, it is necessary that N-bridged polynitrons are used, which exist as polymers and have C—C double and/or C—C triple bonds in the molecule.

For both embodiments, in the curable composition, the ratio of nitron groups (from component a) and unsaturated carbon-carbon bonds (from component b) is 10:1 to 1:10, preferably 5:1 to 1:5, especially 2:1 to 1:2.

In addition to the components (a) and (b) the inventive curable composition may optionally contain the components (c) fillers, and (d) pigments. Further, the composition may comprise one or more (e) additives such as plasticizers, stabilizers and photoinitiators. Finally, the curable composition may also include (f) other cross-linking agents.

The components (a) and (b) are usually contained in the inventive composition in an amount of 30 to 100 weight-%, preferably from 40 to 99 weight-%, more preferably from 55 to 99 weight %-, based on the total weight of the composition.

In principle, as fillers (c), all organic and inorganic fillers can be considered, as described for example, in Römpp lexicon, Lacke und Druckfarben, Georg Thieme Verlag, Stuttgart, New York, 1998, “Füllstoffe”, pages 250 to 252.

Examples of suitable fillers are wood flour, organic or organometallic polymers, inorganic minerals, salts or ceramic materials or organically modified ceramic materials or mixtures of these substances. Inorganic materials are preferably used. These may be natural and synthetic minerals. Examples of suitable minerals are silica, aluminum silicates, calcium silicates, magnesium silicates, calcium aluminum silicates, magnesium aluminum silicates, calcium magnesium silicates, beryllium aluminum silicates, aluminum phosphate or calcium phosphate or mixtures thereof.

In the inventive composition fillers (c) generally are contained in an amount of 0 to 50 weight-%, preferably from 5 to 40 weight-%, more preferably from 10 to 30 weight-%, based on the total weight of the composition.

The inventive composition may further contain as component (d) optionally at least one colorant, preferably a pigment. The colorant may be a pigment or a dye. As pigments, colored pigments or effect pigments can be used. As effect pigments, metal flake pigments such as commercial aluminum bronzes, chromatised aluminum bronzes, commercial stainless steel bronzes, and also nonmetallic effect pigments, such as pearlescent pigments and interference pigments can be used. Reference is made to Römpp lexicon Lacke und Druckfarben, Georg Thieme Verlag, 1998, page 176, Effektpigmente and pages 380 and 381 “Metalloxid-Glimmer-Pigmente” to “Metallpigmente”.

Examples of suitable inorganic chromophoric pigments are titanium dioxide, iron oxides, and carbon black, especially carbon black. Examples of suitable organic chromophoric pigments are thioindigo pigments, indanthrene blue, cromophthal red, irgazine orange and heliogenegrun, copper phthalocyan. Reference is made to Römpp lexicon Lacke und Druckfarben, Georg Thieme Verlag, 1998, pages 180 and 181, “Eisenblau-Pigmente” to “Eisenoxidschwarz”, pages 451 to 453 “Pigmente” to “Pigmentsvolumenkonzentration”, page 563, “Thioindigo Pigmente”, and page 567 “Titandioxid-Pigmente”.

In the inventive composition colorants, preferably pigments (d) generally are contained in an amount of 0 to 30 weight-%, preferably from 1 to 20 weight-%, more preferably from 2 to 10 weight-%, based on the total weight of the composition.

In addition, the inventive composition comprises at least one additive (e). Examples of suitable additives are additional oligomers and polymeric binders, catalysts, scavengers, thermolabile free-radical initiators, radical or cationic photo-initiators, polymerization inhibitors, discharge agents, primers, reactive diluents, flow aids, flow control agents, abrasion and scratching resistant additives, anti-settling agents, antifloating agents, anti-caking additives, anti-blocking additives, antiflocculating agents, deflocculant agents, anti-gelling agents, anti-cratering agents, anti-chalking agents, anti-mottling additives, antioxidants, anti-popping additives, anti-foaming agents, emulsifiers, defoamers, anti-scrape agents, scratch resistance, anti-silking additives, anti-slip agents, antistatic agents, armoring additives, bactericides, fungicides, rottenness preservatives, accelerators, chelating additives chemical resistance improvers, decontaminants, dispersants and grinding aids, emulsifiers, degassing agents, air vent agents, moisture binders, film formers, flame retardants, flow and leveling improvers, formaldehyde reducers, free-flow additives, gloss improvers, lubricants, slip agents, anti-size compounds, surface active agents, coupling agents, curing agents, anti-skinning agents, heat-resistant additives, water repellents, water repellents, catalysts, coalescence aid, coupling reagent, anti-corrosion additives, conductive additives, resistance to solvents, solubilizing agents, air entraining agents, flatting agents, wetting aids, surface improvers, pH control, abrasion resistance improvers, impact modifiers, silver particles, slip additives, barrier additives, special effect additives, stabilizers, separating aids, release additives, release agents, dryers, drying agents, UV absorbers, light stabilizers, thickeners, rheology modifiers, solvents, viscosity reducer, waxes, water retention humectants, emollients and weather-resistance additives.

In the inventive composition, additives (e) are generally contained in an amount of 0 to 20 weight-%, preferably from 0.1 to 10 weight-%, more preferably from 1 to 5 weight-%, based on the total weight of the composition.

In one embodiment it is preferred that the inventive curable composition does not contain catalysts, which catalyze the cross-linking of the unsaturated carbon-carbon bonds in component (b). In an alternative embodiment, the inventive curable composition can contain one or more photoinitiators.

An example of a suitable photoinitiator is Irgacure®.

Photoinitiators can be used in an amount of from 0 to 5 weight-%, preferably from 0.01 to 3 weight-%, more preferably 0.4 to 2.0 weight-%, based on the total weight of the composition.

The inventive composition may as component (e) further optionally include at least one further cross-linking agent. This cross-linking agent can be, for example, a polyisocyanate, a Polyoxaziridin, a polyepoxide, a polyol, a polyphenol, a polyamine or acid anhydrides.

Examples of suitable cross-linking agents are triglycidyl isocyanurate, diglycidylterephtalate, triglycidyl trimellitate, glycidyl methacrylate, caprolactam blocked isophorone diisocyanate derivatives, divinylbenzene, isophorone uretdiones, toluene diisocyanate derivatives, meta- and para-tetramethylxylenediisocyanate, methylenedianiline, 1,3,5-tris(isocyanatomethyl)cyclohexane, 1,3,5-tris(isocyanato) benzene, dicyandiamide derivatives, methylol phenols, trimellitic anhydride, pyromellitic dianhydride, melamine, benzoguanimine, glycoluryl, tris(alkoxycarbonylamino)triazine, N,N,N′,N′-tetrakis(2-hydroxyethyl)adipamide and N,N,N′,N′-tetrakis(2-hydroxypropyl)adipamide.

In the inventive composition the additional cross-linking agents (e) are generally contained in an amount of 0 to 30 weight-%, preferably from 1 to 20 weight-%, more preferably from 2 to 10 weight-%, based on the total weight of the composition.

The inventive curable composition is preferably used as a coating, thin or thick film, adhesive, rubber, filler, sprayable thick layer filler, laminating, storage, casting resin, insulating, sealing, or packing material, ink, printing ink, electric dip paint, radiation-curable coatings, alkyd resin, fiber, paint, foil, powder coating, waterborne paint or solvent based paint. The invention therefore also provides a coating, an adhesive, a rubber, a filler, a sprayable thick film filling, a laminating, storage, or casting resin, an insulating, sealing, or packing material, an ink, a print ink, fiber, a film, an electric dip paint, a radiation-curable coating, an alkyd resin, a powder coating, a waterborne paint or solvent containing paint comprising the composition according to the invention. Preferably, the inventive composition is in the form of a paint, in particular a powder coating material.

The inventive curable composition may be processed by curing (i.e. by cross-linking) into a cross-linked product. Curing (i.e. the cross-linking) is performed by suitable tempering of the curable composition, such as by using electric heating, IR, UV or MW oven.

The invention therefore also provides a process for producing a cross-linking product, comprising the steps of

(i) providing an inventive curable composition and

(ii) curing the composition at temperatures of 20 to 220° C., especially from 20 to 180° C., preferably from 50 to 150° C., particularly preferably from 60 to 120° C.

The present invention also refers to a cross-linking product obtainable by the method according to the present invention.

The curing/cross-linking can be performed, in that the components of the curable composition are mixed and heated. The unsaturated polymer (b) and the N-bridged polynitron (a) (or alternatively an unsaturated polynitron terminated polymer as 1K-system) may, for example milled and mixed in a conventional mill (optionally together with components (c)-(f)) to a powder. Another possibility of mixing is by means of a solvent system, wherein both the unsaturated polymer and the N-bridged polynitrons (optionally together with components (c)-(f)) are dissolved or dispersed. The ingredients are first converted into a uniform mixture, and after removal of the solvent, the curing/cross-linking takes place by heating to the desired temperature.

In the inventive method, the curing time is usually from 10 seconds to 2 hours, preferably from 20 seconds to 60 minutes, more preferably 30 seconds to 15 minutes, more preferably 1 minute to 10 minutes.

The inventive cross-linking products are usually dependent on the type of the unsaturated polymer used. Preferable are elastic-soft to hard cross-linking products. These products are preferably inert to water and organic solvents.

The inventive cross-linking products are versatile. Examples include motor vehicle tires, fiber composite plastics, membranes, tubes, dental materials, home appliances, kitchen countertops, general construction, structural components, bathtubs, safety helmets, sinks, medical devices such as implants, adhesive tapes, adhesives, panels such as boat hulls, fiberglass-reinforced polyester parts and household items. Preferably, the inventive cross-linking products are used as paint layer.

Beneath the use according to the present invention, the inventive curable composition and the inventive cross-linking product, also the preferable N-bridged polynitrons as such are subject-matter of the invention, in particular N-bridged polynitrons made of polyesters, polyamides, and/or aliphatic, cycloaliphatic or aromatic amines having a functionality of primary amine groups of 2 and higher, preferably from 2 to 20 and especially 3 to 6.

The invention also relates to N-bridged polynitrons as they are exemplified in Table I. A person skilled in the art will recognize the relationship between the general structure (I) and the more individualized structures of items, wherein V, w, X¹, Y and Z are as defined above. The radicals R¹ and R² may be, in general, hydrogen, deuterium, an aliphatic, cycloaliphatic or aromatic radical or a polymer chain.

The substituents at the nitron group preferably form a ring for their part. Particularly preferably, R¹ and R² form a ring (in particular a cyclohexane, cyclopentane-, fluorene-, cyclohexa-2,4-dienone, cyclohexa-2,5-dienone, anthracene-10-one, phenanthrene-9-one, naphthalene-1-one, naphthalene-2-one ring). More preferably, R¹ and R² are independently hydrogen, an aryl group (especially phenyl, naphthyl), a heteroaryl group (especially pyridinyl, furyl, thienyl, thiazolyl or benzothiazolyl) or an alkyl group (such as methyl).

Particularly preferably, X¹ contains one or more groups selected from methylene, oxygen, sulfur, carbonyl, sulfone, alkylene, arylene, and the following groups. Y is a natural number larger than one, preferably six or four.

Preferred intermediate groups Z are derived as described above from any substituted, mono- or polynuclear aromatic, cycloaliphatic groups, or alkyl chains.

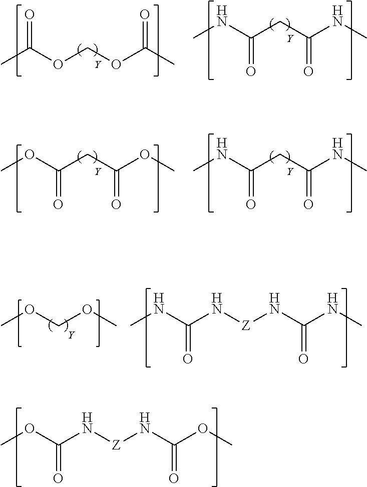

TABLE 1
Exemplary nitrogen-bridged polynitrons of structure (I)

| Ex. | Structure |
|---|---|
| 1 | 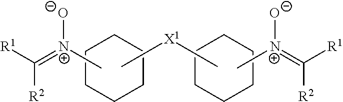 |
| 2 | 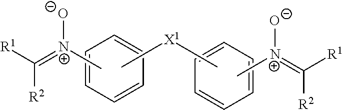 |
| 3 | 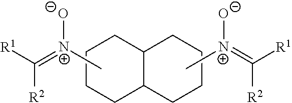 |
| 4 | 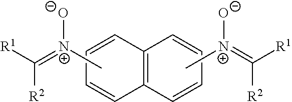 |
| 5 | 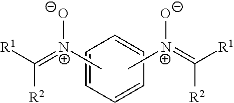 |
| 6 | 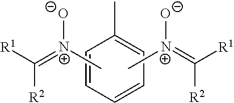 |
| 7 | 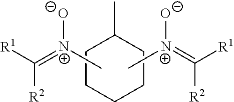 |
| 8 | 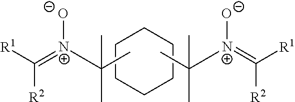 |
| 9 | 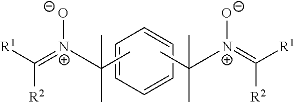 |
| 10 | 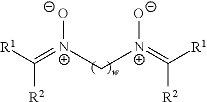 w is a natural number greater than or equal to zero, preferably six. |
| 11 | 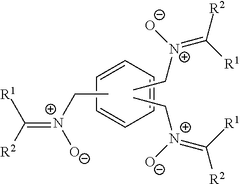 |
| 12 | 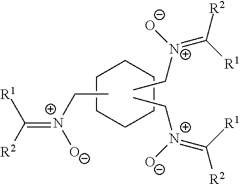 |
| 13 | 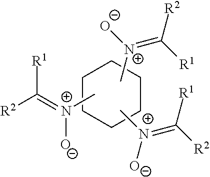 |
| 14 | 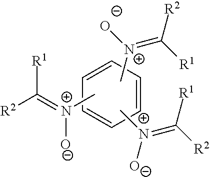 |
| 15 | 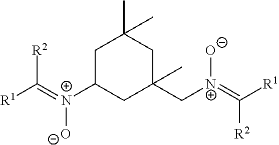 |
| 16 | 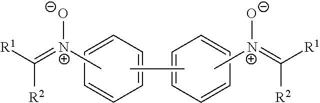 |
| 17 | 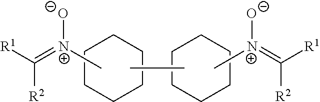 |
| 18 | 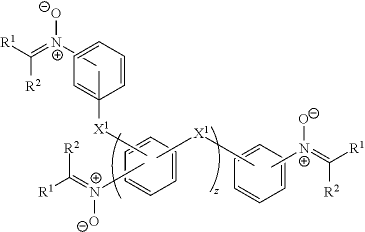 z is a natural number greater than or equal to 1. |
| 19 | 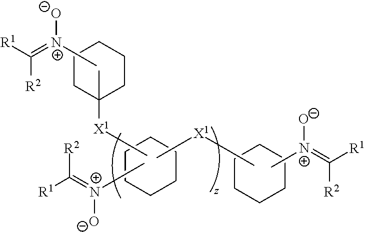 z is a natural number greater than or equal to 1. |
| 20 | 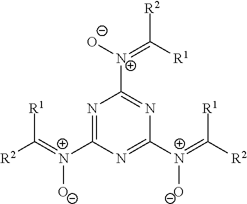 |
| 21 | 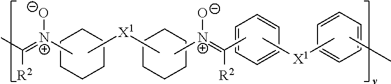 |
| 22 | 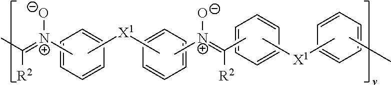 |
| 23 | 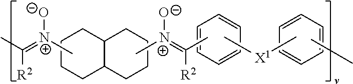 |
| 24 | 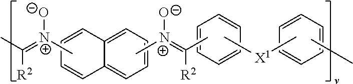 |
| 25 | 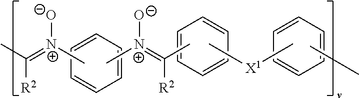 |
| 26 | 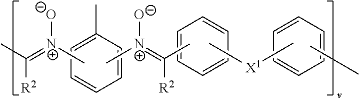 |
| 27 | 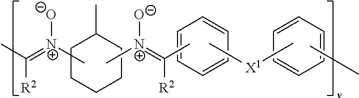 |
| 28 | 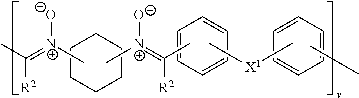 |
| 29 | 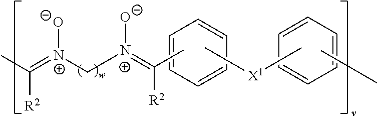 w is a natural number greater than or equal to zero, preferably six |
| 30 | 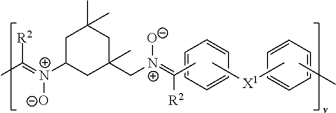 |
| 31 | 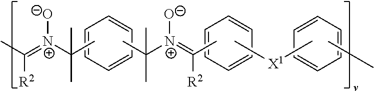 |
| 32 | 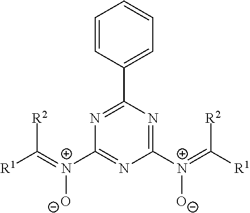 |
| 33 | 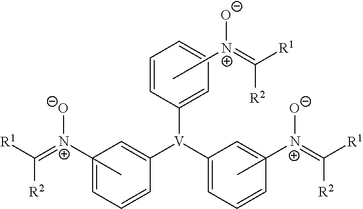 V is particularly preferably a group seleceted from methine, nitrogen, phosphorous, phosphine oxide. |
| 34 | 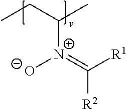 |

In summary, it should be noted, that by the inventive use of N-linked polynitrons unsaturated polymers can be cured at low temperatures. This results in mechanistically stable polymer networks, wherein usually neither harmful metal catalysts are used nor environmentally harmful decomposition products are formed. Cross-linking occurs quickly and provides thermally and mechanistically stable products. An advantage is that N-bridged polynitrons are available in various modifications, have a broad solubility profile and provide a cost effective alternative to carbon-bridged polynitrons. They are available for instance from polyimines, said polyimines firstly may be prepared from primary diamines and aldehydes or ketones. All synthesis steps are efficiently and cost effectively carried out at room temperature under mild conditions in a commercial scale. It is advantageous that diamines and carbonyl compounds are cheaply available in countless variations and thus result in almost unlimited variation possibilities of the polynitron structure. Due to this structure diversity, the properties of the polynitrons such as melting point and those of the cross-linking products such as glass transition temperature are almost arbitrarily controllable.

The produced cross-linking products according to the invention are characterized by their versatility at relatively low cost in manufacture. They are easy to handle, can be used alone or optionally together with minor amounts of other polymers and can be processed with a large number of fillers.

The inventive cross-linking method provides new opportunities for the development of new materials for an environmentally friendly option. The combination of an unsaturated polymer and N-bridged polynitrons can be used in the following areas for the development of new products:
1 Development of new adhesives (e.g., hot melt adhesives, acrylic adhesives);
2 Use as laminating, bonding, storage, casting and paint resin;
3 In the furniture sector: through fast curing and virtually 100% solid body the application of very thick layers is possible in a single pass;
4 Production of fast-curing and good sandable fillers (auto repair, woodworking and metalworking industries);
5 Preparation of injectable thick layer fillers (car repair, woodworking and metalworking industries);
6 Production of glass fiber reinforced polyester parts (glass fiber reinforced polyester), for example in shipbuilding;
7 Development of new waterborne paints, powder paints and solvent based paints;
8 Development of self-curing powder paints (nitron terminated unsaturated polymers);
9 Development of insulating, sealing, and packaging material; Use for impregnation;
10 Use for producing large panels such as boat hulls;
11 Development of new fibers and films, and textiles;
12 Use for protective helmets and fittings;
13 Development of new medical devices such as implants;
14 Use for household items and electrical engineering;
15 Development of new printing inks and inks, in particular inkjet inks;
16 Use in photocationic polymerizations, for example, in lithography.

### EXAMPLES

### Example 1

### Synthesis of N,N′-bis(9-fluorenylidene)-4,4′-diaminodiphenylmethane-N,N′-dioxide (PN-1)

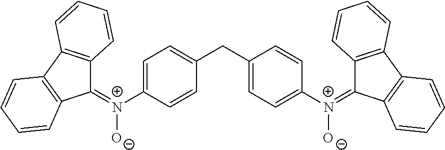 [SMILES: [O-][N+](=C1c2ccccc2-c2ccccc21)c1ccc(Cc2ccc([N+]([O-])=C3c4ccccc4-c4ccccc43)cc2)cc1]

9.00 g (50.0 mmol) of 9-fluorenone are dissolved in 150 mL of toluene and mixed with a solution of 14.8 g (75.0 mmol) of 4,4′-diaminodiphenylmethane in 100 mL of toluene. Within one hour, 5.70 g (30.0 mmol) of titanium (IV) chloride are added dropwise to 50 mL of toluene under cooling in an ice bath. After 24 hours stirring at room temperature the resulting solid is separated by filtration and toluene is removed by distillation from the organic phase. The resulting crude product is recrystallized in ethanol and dissolved in 50 mL of dichloromethane. While cooling in an ice bath 8.63 g (50.0 mmol) of meta-chloroperbenzoic acid is slowly added dropwise to 50 mL dichloromethane. The solution is cooled for two hours in an ice bath and then the resultant meta-chlorobenzoic acid is removed by filtration. The organic phase is washed twice with 50 mL of aqueous sodium sulfite solution (1M), washed once with 50 mL aqueous sodium bicarbonate solution (2M) and washed twice with 50 mL water and then dried over magnesium sulfate. The solid is separated by filtration and the solvent is removed under reduced pressure. Melting point: about 240° C.

### Example 2

### Synthesis of N,N′-dibenzylidene hexan-1,6-diamine-N,N′-dioxide (PN-2)

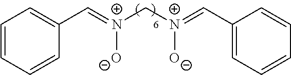

5.0 g (43 mmol) of 1,6-diaminohexane, 9.1 g (86 mmol) of benzaldehyde and 10 g of magnesium sulfate are stirred for 24 hours in 50 mL of methanol at room temperature. The solvent is removed under reduced pressure and the residue is stirred in 50 mL dichloromethane. Magnesium sulfate is removed by filtration. While cooling in an ice bath, 14.8 g (86.0 mmol) of meta-chloroperbenzoic acid in 50 mL of dichloromethane is slowly added dropwise to the organic phase. The solution is stirred for two hours in an ice bath and then the resultant meta-chlorobenzoic acid is removed by filtration. The organic phase is washed twice with 50 mL of aqueous sodium sulfite solution (1M), washed once with 50 mL aqueous sodium bicarbonate solution (2M) and washed twice with 50 mL water and then dried over magnesium sulfate. The solid is separated by filtration and the solvent is removed under reduced pressure. Melting point: about 105° C.

### Example 3

### Synthesis of N,N′-bis(4-hexyloxybenzyliden) hexane-1,6-diamine-N,N′-dioxide (PN-3)

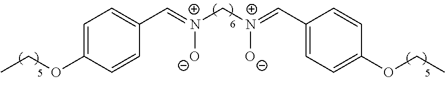

2.8 g (24 mmol) of 1,6-diaminohexane, 10 g (48 mmol) of 4-hexyloxybenzaldehyde and 8 g of magnesium sulfate are stirred for 24 hours in 50 mL of methanol at room temperature. The solvent is removed under reduced pressure and the residue was stirred in 50 mL dichloromethane. Magnesium sulfate is removed by filtration. While cooling in an ice bath, 8.3 g (48 mmol) of meta-chloroperbenzoic acid in 50 mL of dichloromethane is slowly added dropwise to the organic phase. The solution is stirred for two hours in an ice bath and then the resultant meta-chlorobenzoic acid was removed by filtration. The organic phase is washed twice with 50 mL of aqueous sodium sulfite solution (1M), washed once with 50 mL aqueous sodium bicarbonate solution (2M) and washed twice with 50 mL water and then dried over magnesium sulfate. The solid is separated by filtration and the solvent is removed under reduced pressure. Melting point: about 80° C.

### Example 4

### Synthesis of N,N′-dibenzylidene-4,4′-methylene-bis(cyclohexylamine)-N,N′-dioxide (PN-4)

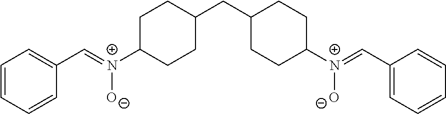 [SMILES: [O-]/[N+](=C\c1ccccc1)C1CCC(CC2CCC(/[N+]([O-])=C/c3ccccc3)CC2)CC1]

5.0 g (43 mmol) of 4,4′-methylene bis(cyclohexylamine), 9.1 g (86 mmol) of benzaldehyde and 1 g of palladium on activated carbon are approximately stirred for 48 hours in 100 mL methanol at room temperature in a hydrogen atmosphere. The catalyst is removed by filtration. Under cooling in an ice bath, initially 0.5 g (4.3 mmol) of selenium dioxide is added to the filtrate and then 29 g (258 mmol) of hydrogen peroxide (30% in water) is slowly added dropwise. The solution is stirred for three hours in an ice bath and then concentrated under reduced pressure. The precipitated white solid is washed several times with water and dried under high vacuum. Melting point: about 155° C.

### Example 5

### Synthesis of N,N′-dibenzylidene-1,4-cyclohexylamine-N,N′-dioxide (PN-5)

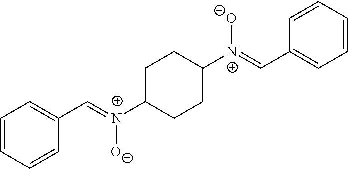 [SMILES: [O-]/[N+](=C\c1ccccc1)C1CCC(/[N+]([O-])=C/c2ccccc2)CC1]

5.0 g (44 mmol) of 1,4-diaminocyclohexane, 9.3 g (88 mmol) of benzaldehyde and 1 g of palladium on activated carbon are stirred for about 48 hours in 100 mL methanol at room temperature in a hydrogen atmosphere. The catalyst is removed by filtration. Under cooling in an ice bath initially 0.5 g (4.4 mmol) of selenium dioxide is added to the filtrate and then 30 g (264 mmol) of hydrogen peroxide (30% in water) is slowly added dropwise. The solution is stirred for three hours in an ice bath and then concentrated under reduced pressure. The precipitated white solid is washed several times with water and dried under high vacuum. Melting point: about 270° C.

### Example 6

### Synthesis of N,N′-(3-pyridinylmethylene)-1,6-diamine-N,N′-dioxide (PN-6)

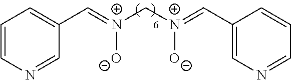

5.0 g (43 mmol) of 1,6-diaminohexane, 9.1 g (86 mmol) of 3-picolylamine and 1 g of palladium on activated carbon are stirred for about 48 hours in 100 mL methanol at room temperature in a hydrogen atmosphere. The catalyst is removed by filtration. Under cooling in an ice bath initially 0.5 g (4.4 mmol) of selenium dioxide is added to the filtrate and then 14.8 g (86.0 mmol) of hydrogen peroxide (30% in water) is slowly added dropwise. The solution is stirred for three hours in an ice bath and then methanol is removed under reduced pressure. The residue is mixed with water and dichloromethane. The separated organic phase is washed twice with 50 mL of aqueous sodium sulfite solution (1M), washed once with 50 mL aqueous sodium bicarbonate solution (2M) and washed twice with 50 mL water and then dried over magnesium sulfate. The solid is separated by filtration and the solvent is removed under reduced pressure. Melting point: about 95° C.

### Example 7

### Synthesis of N,N′-dibenzylidene[poly-N,N′-terephtalyliden-4,4′-methylene bis(cyclohexylamine)]-N,N′-polyoxide) (PN-7)

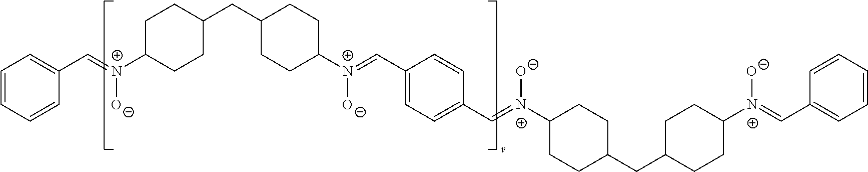

5.0 g (37 mmol) terephtalic dialdehyde, 11.8 g (55.9 mmol) of 4,4′-methylene-bis (cyclohexylamine), 3.9 g (37 mmol) of benzaldehyde and 1 g of palladium on activated carbon are stirred for about 48 hours in 200 mL of methanol at room temperature in a hydrogen atmosphere. The catalyst is removed by filtration. Under cooling in an ice bath, initially 1.23 g (11.1 mmol) of selenium dioxide is added to the filtrate and then 75.5 g (666 mmol) of hydrogen peroxide (30% in water) is slowly added dropwise. The solution is stirred for five hours in the ice bath and then concentrated under reduced pressure. The precipitated white solid is washed several times with water and dried under high vacuum. Melting range: about 190 to 240° C.

### Example 8

### Cross-Linking of Unsaturated Polyester Uracross P3125 with PN-1 PN-2 and PN-3

10 to 20 wt % nitrogen-bridged polynitron PN-1 to PN-8 are heated at 120° C. for 30 to 120 minutes with 80 to 90 weight-% Uracross P3125. Uracross P3125 is a commercial product of the company DSM and axhibits unsaturated maleate or fumarate units. By storing for one hour in the oven at 120° C., a solvent-resistant cross-linked material has been formed.

The cross-linking can be recognized by a change in the glass transition temperature of Uracross P3125, which were examined by means of DSC measurements (differential scanning calorimetry) on a Mettler Toledo DSC822 in a temperature range from −50° C. to 220° C. at a heating rate of 10° C./min. The glass transition temperatures were expressed as mean values from the second and third heating process, wherein the temperature in each case was given, at which half of the heat capacity change was achieved. For calibration, tin, indium and zinc standards were used.

Non-cross-linked Uracross P3125 has a glass transition temperature of 52° C., which changes by cross-linking. As shown in Table II, the glass transition temperature of the cross-linking product is arbitrarily controlled with the help of the structure of the nitrogen-bridged polynitrons.

TABLE 2
Glass transition temperature of
non-cross-linked Uracross P3125 and with N-bridged
polynitron cross-linked Uracross P3125

| | Glass |
|---|---|
| | transition |
| Polymer | temperature |
| Non-cross-linked Uracross P3125 | 52° C. |
| With PN-1 cross-linked Uracross | 69° C. |
| P3125 | |
| With PN-2 cross-linked Uracross | 62° C. |
| P3125 | |
| With PN-3 cross-linked Uracross | 45° C. |
| P3125 | |
| With PN-4 cross-linked Uracross | 69° C. |
| P3125 | |
| With PN-5 cross-linked Uracross | 57° C. |
| P3125 | |
| With PN-6 cross-linked Uracross | 56° C. |
| P3125 | |
| With PN-7 cross-linked Uracross | 78° C. |
| P3125 | |

It was also confirmed by IR spectroscopy that no unsaturated double bonds are present after the cross-linking.

1. A method for producing polynitrons by oxidation of a polyimine or polyamine.

2. The method according to claim 1, characterized in that the polyimine or polyamine has a functionality of 2 and higher, more preferably of 2 to 20, most preferably of 3 to 6.

3. The method according to claim 1, characterized in that the polynitron is a compound according to general formula (I)
[X—Z(—X)n]m (I),
in which X is a preferably substituted nitron group, Z is a polyfunctional aliphatic, cycloaliphatic, aromatic, substituted or unsubstituted intermediate group, which may also contain heteroatoms and/or may be a polymer and n and m is an integer >0, preferably an integer >1.

4. The method according to claim 1, characterized in that the polyimine used is produced by hydration of a polyimine.

5. The method according to claim 1 characterized in that the polyimine used is prepared by reaction of a polyfunctional aldehyde or ketone with a monofunctional primary amine.

6. The method according to claim 1, characterized in that the polyimine used is prepared by reaction of a polyfunctional primary amine with a monofunctional aldehyde or ketone.

7. The method according to claim 1 characterized in that the polynitron is a N-bridged polynitron.

8. The method according to claim 7, characterized in that the nitron group is N side bridged via a polymer.

9. The method according to claim 7, characterized in that the N-bridged polynitron is prepared from aliphatic, cycloaliphatic and/or aromatic amines having a functionality of primary amine groups of 2 and higher, preferably from 2 to 20 and more preferably from 3 to 6.

10. The method according to claim 1, characterized in that the polynitron is prepared from polymers with primary amino groups, selected from the group, consisting of polyvinyl amines, polyamides, polyurethanes or urea melamine resins.

11. The method according to claim 3, characterized in that the polynitron is a N-bridged polynitron and the intermediate group Z is derived from the following structures: 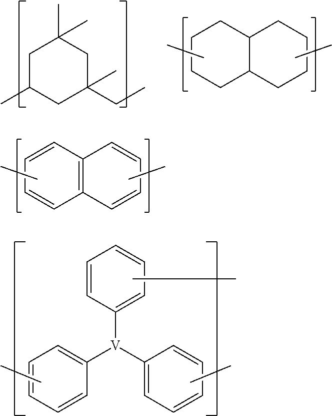
V is a group selected from methine, nitrogen, phosphorous, phosphine oxide, 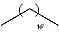
W is a natural number greater than zero, 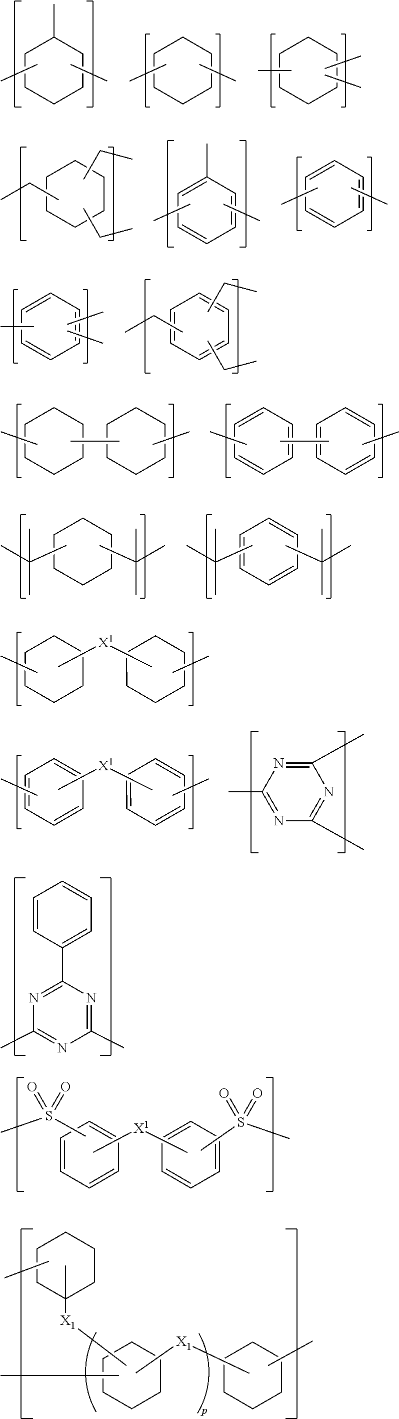 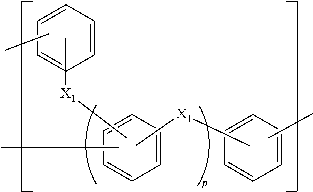
p is a natural number greater than or equal to 1 and X1 is an aliphatic, cycloaliphatic, aromatic, substituted or unsubstituted group which may also contain hetero atoms and/or may be a polymer.

12. Use of N-bridged polynitrons produced by a method according to claim 1 for cross-linking of unsaturated polymers.

13. A curable composition, comprising
(a) an N-bridged polynitron,
(b) an unsaturated polymer or a mixture of polymers, wherein at least one polymer contains unsaturated functions or functional groups which can react with the polynitron,
(c) optionally fillers,
(d) optionally pigments,
(e) optionally additives, preferably plasticizers, stabilizers and/or photoinitiators, and
(f) optionally further cross-linking agents, preferably polyisocyanates, bisdienes and/or polyoxaziridines.

14. The composition according to claim 13, characterized in that the N-bridged polynitron is a compound according to general formula (I)
[X—Z(—X)n]m (I),
in which X is a preferably substituted nitron group,
Z is a polyfunctional aliphatic, cycloaliphatic, aromatic, substituted or unsubstituted intermediate group, which may also contain heteroatoms and/or may be a polymer and is derived from the following structures: 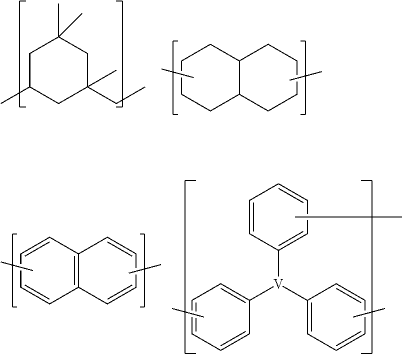
V is a group selected from methine, nitrogen, phosphorous, phosphine oxide, 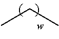
W is a natural number greater than zero, 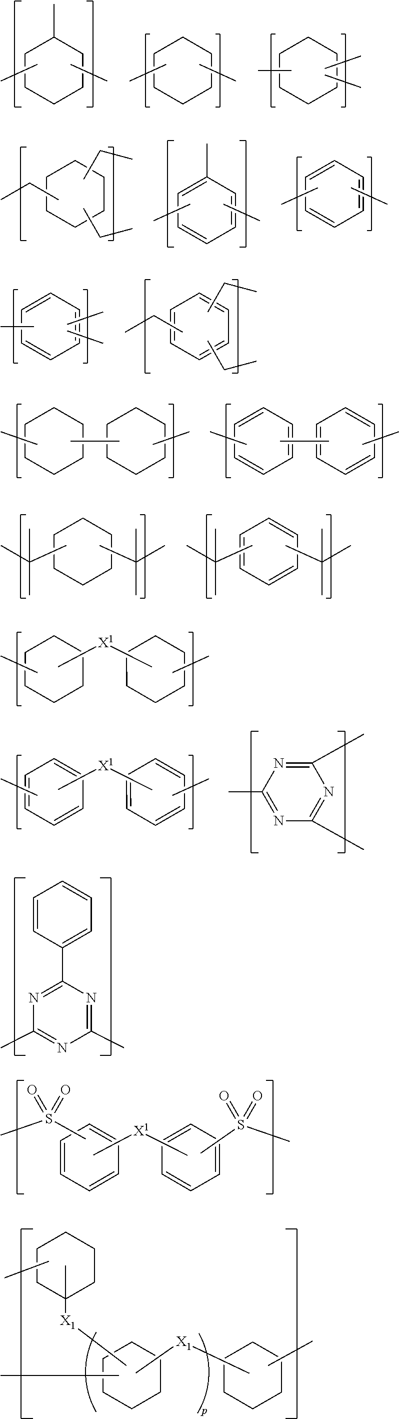 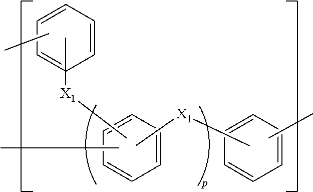
p is a natural number greater than or equal to 1 and X1 is an aliphatic, cycloaliphatic, aromatic, substituted or unsubstituted group which may also contain hetero atoms and/or may be a polymer
and n and m is an integer >0, preferably an integer >1.

15. The composition according to claim 13, characterized in that it is a 2-component system in which the components (a) and (b) are present in the form of two different compounds.

16. The composition according to claim 13, characterized in that it is a 1-component system, wherein components (a) and (b) are present in the form of a polynitron-terminated unsaturated polymer.

17. Use of a composition according to claim 13 as coating, thin or thick film, adhesive, rubber, fiber, film, filler, sprayable thick layer filler, insulating, sealing, packaging material, ink, paint, electric dip coating, radiation-curable coatings, alkyd resin, powder paint, waterborne paint, solvent based paint, laminating, storage, or casting resin, as well as in lithography.

18. A method for producing a cross-linking product, comprising the steps of (a) providing a curable composition according to claim 13, (b) curing the composition at temperatures of 20 to 220° C., preferably from 20 to 180° C.

19. A cross-linking product obtainable by a method according to claim 18.

20. Use of a cross-linking product according to claim 19 for manufacture of motor vehicle tires, fiber composite plastics, membranes, tubes, dental materials, household appliances, kitchen countertops, bathtubs, sinks, structural components, large panels such as boat hulls, medical devices such as implants, protective helmets and fiberglass-reinforced polyester parts.
